(19) United States
(12) Patent Application Publication
(10) Pub. No.: US 2010/0178364 A1
(43) Pub. Date: Jul. 15, 2010

(54) Dietary supplements against latent foreign DNA

(76) Inventor: Hanan Polansky, Rochester, NY (US)

Correspondence Address: Hanan Polansky, 2030 Elmwood Ave., Rochester, NY, 14620, US

(21) Appl. No.: 12/351,907

(22) Filed: Jan. 12, 2009

(51) Int. Cl. A61K 33/04; A61K 36/484; A61K 31/19; A61K 31/352; A61K 36/82; A61K 36/54; A61K 31/11; A61K 31/192; A61K 31/335; A61P 37/00

(52) U.S. Cl. 424/702; 424/757; 514/557; 514/456; 424/729; 424/739; 514/701; 514/570; 514/450

(57) ABSTRACT

The invention presents methods for decreasing the concentration of a foreign polynucleotide in a human or animal subject, especially, when such polynucleotide is latent in the subject.

### I. BACKGROUND OF THE INVENTION

Currently, the medical community considers latent foreign DNA as harmless. For instance, consider the following definitions of a latent virus. All definitions were found on the internet on Dec. 24, 2008:

According to The Dictionary of Cell & Molecular Biology, (Paperback), by John Lackie (Author), Academic Press; 4 edition (9 Jun. 2007): (A latent virus is a) “Virus integrated within host genome but inactive: may be reactivated by stress such as ultraviolet irradiation.” Note the word “inactive” suggesting harmless. According to this definition, the latent virus becomes dangerous only upon activation, that is, upon turning lytic.

According to Wikipedia: “While viral latency exhibits no active viral shedding nor causes any pathologies or symptoms, the virus is still able to reactivate via external activators (i.e. sunlight, stress) to cause an acute infection. In the case of Herpes simplex virus, which generally infects an individual for life, a serotype of the virus reactivates occasionally to cause cold sores. The sores are quickly resolved by the immune system, however may be a minor annoyance from time to time. In the case of varicella zoster virus, after an initial acute infection (chickenpox) the virus lies dormant until reactivated as herpes zoster.” Note the phrase: “nor causes any pathologies or symptoms” and the phrase “dormant until reactivated,” both suggesting being harmless until reactivated.

According to WrongDiagnosis.com: “Latent virus infection: phase during the course of a viral infection during which the pathogens are dormant or have not yet produced symptoms.” Note the phrase: “dormant or have not yet produced symptoms.”

According to CRISP the Sci-Tech Dictionary: “Latent virus (virology): A virus that remains dormant within body cells but can be reactivated by conditions such as reduced host defenses, toxins, or irradiation, to cause disease.” Note the phrase “dormant within body cells but can be reactivated . . . to cause disease.”

According to the Gale Encyclopedia of Medicine, as quoted in the medical-dictionary.thefreedictionary.com: “Latent virus: A nonactive virus which is in a dormant state within a cell. Herpes virus is latent in cells of the nervous system.” Note the phrase “A nonactive virus which is in a dormant state within a cell”

These sources suggest that a person skilled in the art will consider a latent virus, or latent foreign DNA, in 2008, as harmless.

However, in 2003, a study showed that microcompetition between common foreign polynucleotides and certain cellular genes for a limiting transcription complex causes many chronic diseases, see Polansky 2003¹ and U.S. Pat. No. 7,381,526², hereby expressly and entirely incorporated by reference. As a result of this discovery, the current invention presents methods for decreasing foreign DNA in the host, especially, when the DNA is latent.

### II. BRIEF SUMMARY OF THE INVENTION

In one respect, the invention presents methods for preventing or attenuating microcompetition between a foreign polynucleotide and a cellular polynucleotide or attenuating an effect of such microcompetition. For example, to ameliorate a disease symptom resulting from microcompetition between a foreign polynucleotide and a cellular polynucleotide, a dietary supplement can be administered to the host to reduce the cellular copy number of the foreign polynucleotide, to reduce the formation of a complex between the foreign polynucleotide and a cellular transcription factor, to increase the formation of a complex between the microcompeted cellular transcription factor and the cellular polynucleotide, or to reverse an effect of microcompetition on the expression or activity of a polypeptide with expression regulated by the cellular polynucleotide, directly, or indirectly through, for instance, increasing the effectiveness of the immune system, or modifying a function of the immune system.

In a preferred embodiment, the methods feature administrating an effective dose of a dietary supplement to a human or animal subject for decreasing the concentration of a foreign polynucleotide, especially when such polynucleotide is latent in the subject.

### III. DETAILED DESCRIPTION OF THE INVENTION

The following sections present descriptions of elements used in the present invention. The descriptions include a definition and one or more exemplary assays to teach one skilled in the art on how to use the element. Each assay may include, as its own elements, standard methods in molecular biology, microbiology, cell biology, cell culture, transgenic biology, recombinant DNA, immunology, pharmacology, and toxicology, well known in the art. Descriptions of other elements can found in Polansky 2003 (ibid), U.S. Pat. No. 7,381,526(ibid), and patent application Ser. No. 12/335,576³, hereby expressly and entirely incorporated by reference.

### A. Latent and Persistent Foreign Polynucleotides

### Definition

Consider a polynucleotide foreign to a given organism. The polynucleotide will be called latent in a cell of the organism if over an extended period of time, either:
1. The polynucleotide produces no transcripts.
2. The set of gene products expressed by the polynucleotide in the cell is a subset of all possible gene products of that polynucleotide.
3. The polynucleotide shows limited or no replication.
4. The polynucleotide is undetected by the host immune system, or is detected but is not completely cleared and is maintained in a certain reservoir.
5. The cell shows no lytic symptoms.
6. The organism shows no macroscopic symptoms.

### Notes:

1. A virus in a host cell is a foreign polynucleotide. According to the definition, a virus is considered latent if, over an extended period of time, it either shows partial expression of its gene products, shows no viral mRNA, shows limited or no replication, is undetected by the host immune system, is maintained in a certain reservoir, causes no lytic symptoms in the infected cell, or causes no macroscopic symptoms in the host.
2. A latent polynucleotide is sometimes called an asymptomatic polynucleotide.
3. There a several common methods used by persons skilled in the art to detect and characterize latent infections of common viruses. Two such methods are serology and PCR, either qualitative or quantitative. For instance, a common method to detect and characterizing a latent EBV infection is to assay the presence of antibodies to the viral VCA or EBNA. The VCA assay is also called VCA IgG, or EBV IgG. Another standard method for detecting and characterizing a latent EBV infection is to assay the EBV viral load and/or EBV viremia using real time PCR. During latency, the results of these assays are correlated. For instance, a study⁴ showed a positive correlation between EBV viral load and VCA IgG titers in Hodgkin's Lymphoma patients and their healthy relatives. Another study⁵ showed a positive correlation between EBV viral load and VCA and EA IgG titers in healthy subjects. The study also showed a positive correlation between CMV viral load and CMV antibody titers in healthy subjects. A third study⁶ showed a positive correlation between proviral load and anti-Env antibodies in asymptomatic HTLV-I carriers.
4. Some of the viruses that establish latency in humans, either in the form of proviral or episomal latency, include the Epstein Barr Virus (EBV), Cytormegalovirus (CMV), Herpes Simplex Virus 1 (HSV-1), Herpes Simplex Virus 2 (HSV-2), Human Papillomavirus (HPV), Adenovirus, Kaposi's Sarcoma-Associated Herpesvirus (KSHV, also called Human Herpesvirus 8, HHV-8), Hepatitis B, Torquetenovirus (TTV, also called Transfusion Transmitted Virus), JC Virus (JCV), BK Virus (BKV), and Retroviruses.
5. The above list of characterizations is not exhaustive. The medical literature includes more aspects of latency that can be added to the definition.

### Exemplary Assays

1. Introduce, or identify a foreign polynucleotide in a host cell. Assay the polynucleotide replication, or transcription, or mRNA, or gene products over an extended period of time. If the polynucleotide shows limited replication, no transcription, or a limited set of transcripts, the polynucleotide is latent.
2. Introduce, or identify a foreign polynucleotide in a host cell. Assay the cell over an extended period of time, if the cell shows no lytic symptoms, the polynucleotide is latent.

### EXAMPLES

Using PCR, a study (Gonelli 20017) observed persistent presence of viral human herpes virus 7 (HHV-7) DNA in biopsies from 50 patients with chronic gastritis. The study also observed no U14, U17/17, U31, U42 and U89/90, HHV-7 specific transcripts highly expressed during replication. Based on these observations, the study concluded that “gastric tissue represents a site of HHV-7 latent infection and potential reservoir for viral reactivation.” To test the effect of treatment on the establishment of latent herpes simplex virus, type 1 (HSV-1) in sensory neurons, another study (Smith 20018) assays the expression of the latency-associated transcript (LAT), the only region of the viral genome transcribed at high levels during the period of viral latency. A review (Young 20009) discusses the limited sets of Epstein-Barr viral (EBV) gene products expressed during the period of viral latency.

See more explanations and examples in U.S. Pat. No. 7,381,526 (ibid)

Each individual host has a latency set point, or small range, at which the immune system and viral factors that control the level of latency are in equilibrium (Virgin 2005¹⁰). According to Virgin 2005 (ibid), “There are good data that persistent replication occurs in both normal and immunocompromised mice and that it can contribute to the pool of latently infected cells.” Also, according to Virgin 2005 (ibid), “In addition, persistent replication can significantly contribute to maintenance of a pool of latently infected cells, perhaps via infection of new cells in which the virus can establish latency. For example, in B-cell-deficient mice both persistent replication and increased number of latently infected cells are observed. If these mice are treated with either an antiviral Ab specific for a lytic cycle viral antigen or an antiviral drug that targets the viral DNA polymerase, the number of latently infected cells diminishes dramatically. This is most consistent with the role for persistent γHV68 replication as a major contributor to the size of the latently infected cell pool.” Also consider Gangappa 2002¹¹: “In the experiments reported here, we determined the effect of passively transferred antibody on established γHV68 latency in B-cell-deficient (B-cell(−/−)) mice. Immune antibody decreased the frequency of cells reactivating ex vivo from latency in splenocytes (>10-fold) and peritoneal cells (>100-fold) and the frequency of cells crying latent viral genome in splenocytes (>5-fold) and peritoneal cells (>50-fold). . . . Passive transfer of antibody specific for the lyric cycle γHV68 RCA protein decreased both the frequency of cells reactivating ex vivo from latency and the frequency of cells carrying the latent viral genome. Therefore, antibody specific for lytic cycle viral antigens can play an important role in the control of gammaherpesvirus latency in immunocompromised hosts. Based on these findings, we propose a model in which ongoing productive replication is essential for maintaining high levels of latently infected cells in immunocompromised hosts. We confirmed this model by the treatment of latently infected B-cell(−/−) mice with the antiviral drug cidofovir.” Also “antibody to lytic cycle can decrease the frequency of latently infected cells by breaking a cycle in which lytic replication contributes to the pool of latently infected cells in B-cell(−/−) mice.” . . . “This in turn suggests that ongoing or sporadic productive infection plays an important part in the maintenance of high frequencies of latently infected cells in B-cell(−/−) mice. Consistent with this model, inhibition of productive infection with an antiviral drug also decreased the frequency of latently infected cells.” See also Virgin 2005 (ibid): “Finally, there is excellent evidence that the immune system is important for maintaining a normal level of latently infected cells and for regulating the efficiency of reactivation from latency. In addition, the frequency of cells latently infected with EBV is significantly increased in immunocompromised patients.” See also Hosino 2008¹² and Kim 2002¹³.

These studies suggest that any disruption of persistent replication or reactivation from latency diminishes the number of latently infected cells or the virus copy number in latently infected cells, together defining the latent viral load.

Also, according to Virgin 2005 (ibid): “Several groups have tested the hypothesis that high levels of preexisting immunity might attenuate chronic γ-herpesvirus disease by limiting infection. Vaccination against γHV68 infection with single viral antigens attenuates acute infection and decreases the amount of latent infection at early time points (2 to 3 wk infection). For example, vaccination against the major membrane glycoprotein gp150 induced a neutralizing Ab response and reduced the number of latently infected cell 14 d after infection). Similarly, T-cell vaccination using immunodominant CD8 T-cell epitopes derived from lytic-cycle antigens decreased both acute titer and latency at d 14 after infection, and CD8 T-cells specific for the latent viral antigen decreased latency early after infection. Despite the success of these approaches in the control of acute and early latent infection, these approaches failed to produce a detectable change in long-term latency (d 28 after infection and beyond).”

These observations suggest that a decrease in viral load during the acute phase and early latent phase of the infection is not necessarily indicative of the viral load during the established latent phase.

### SUMMARY

Microcompetition between a foreign polynucleotide and a cellular gene for a limiting transcription complex results in aberrant transcription of the cellular gene. Aberrant transcription results in disease. Therefore, microcompetition between a foreign polynucleotide and a cellular gene for a limiting transcription complex results in disease. When the foreign polynucleotide persists in the host cells or is latent in the host cells for an extended period of time, microcompetition between the foreign polynucleotide and the cellular gene results in a chronic disease. Chronic diseases caused by microcompetition with a foreign polynucleotide include atherosclerosis, cancer, obesity, osteoarthritis, type II diabetes, type I diabetes, multiple sclerosis, asthma, lupus, thyroiditis, inflammatory bowel disease, rheumatoid arthritis, psoriasis, atopic dermatitis, graft versus host disease, and other autoimmune diseases (see Polansky 2003, ibid).

### B. Treatment with Dietary Supplements

1. Introduction

In one aspect, the invention presents methods for treating chronic diseases resulting from the presence of latent viral genomes in a host. In a preferred embodiment, the methods feature administration to a subject an effective dose of a dietary supplement to decrease the concentration of the latent viral load, prevent the load from increasing, or diminish the rate that this load is increasing. The following examples demonstrate that treatment with dietary supplements can decrease the copy number of latent viral genomes in infected cells, the number cells with latent infection, and the latent viral copy number in the cells. These examples demonstrate that dietary supplements can decrease the concentration of foreign polynucleotides in cells, and as a result increase in availability of limiting transcription complex to cellular genes, and therefore, diminish the deleterious effect of the foreign polynucleotide on transcription, cell behavior, and the host's health.

2. Treatment Protocols

a) Introduction

In one aspect, the invention presents methods for treating chronic diseases. In a preferred embodiment, the methods feature administration to a subject an effective dose of a dietary supplement that prevents or attenuates microcompetition between a foreign polynucleotide and a cellular polynucleotide or attenuates an effect of such micocompetition. For example, if the cellular polynucleotide is a GABP regulated gene, the dietary supplement can reduce the copy number of the foreign polynucleotide, stimulate the expression of the GABP regulated gene, increase the bioactivity of the GABP regulated gene, through, for instance, phosphorylation of GABP and/or increasing the bioavailability of a GABP regulated protein, through, for instance, a reduction in copy number of the foreign polynucleotides which bind GABP. A dietary supplement can also, for example, can inhibit the expression a cellular gene that increases expression as a result of microcompetition with a foreign polynucleotide, such as, tissue factor, androgen receptor, and/or inhibit replication of a p300/cbp virus.

The following sections describe standard protocols for determining effective dose, and for agent formulation for use. Additional standard protocols and background information are available in books, such as In vitro Toxicity Testing Protocols (Methods in Molecular Medicine, 43), edited by Sheila O'Hare and C K Atterwill, Humana Press, 1995; Current Protocols in Pharmacology, edited by: S J Enna, Michael Williams, John W Ferkany, Terry Kenakin, Roger D Porsolt, James P Sullivan; Current Protocols in Toxicology, edited by: Mahin Maines (Editor-in-Chief), Lucio G Costa, Donald J Reed, Shigeru Sassa, I Glenn Sipes; Remington: The Science and Practice of Pharmacy, edited by Alfonso R Gennaro, 20^{th} edition, Lippincott, Williams & Wilkins Publishers, 2000; Pharmaceutical Dosage Forms and Drug Delivery Systems, by Howard C Ansel, Loyd V Allen, Nicholas G Popovich, 7^{th} edition, Lippincott Williams & Wilkins Publishers, 1999; Pharmaceutical Calculations, by Mitchell J Stoklosa, Howard C Ansel, 10^{th} edition, Lippincott, Williams & Wilkins Publishers, 1996; Applied Biopharmaceutics and Pharmacokinetics, by Leon Shargel, Andrew B C Yu, 4^{th} edition, McGraw-Hill Professional Publishing, 1999; Oral Drug Absorption: Prediction and Assessment (Drugs and the Pharmaceutical Sciences, Vol 106), edited by Jennifer B Dressman, Hans Lennemas, Marcel Dekker, 2000; Goodman & Gilman's The Pharmacological Basis of Therapeutics, edited by Joel G Hardman, Lee E Limbird, 10^{th} edition, McGraw-Hill Professional Publishing, 2001. See also above referenced.

b) Effective Dose

Compounds can be administered to a subject, at a therapeutically effective dose, to treat, ameliorate, or prevent a chronic disease. Monitoring of patient status, using either systemic means, standard clinical laboratory assays, or assays specifically designed to monitor the bioactivity of such compounds on the foreign polynucleotide and the foreign polynucleotide's effects, can be used to establish the effective therapeutic dose and to monitor this effectiveness.

Prior to patient administration, techniques standard in the art may be used with any agent described herein to determine the LD₅₀ and ED₅₀ (lethal dose which kills one half the treated population, and effective dose in one half the population, respectively) either in cultured cells or laboratory animals. The ratio LD₅₀/ED₅₀ represents the therapeutic index which indicates the ratio between toxic and therapeutic effects. Compounds with a relatively large index are preferred. These values are also used to determine the initial therapeutic dose.

c) Formulation for Use

Those skilled in the art recognize a host of standard formulations for the agents described in this invention. For instance, the agent may be given orally by delivery in a tablet, capsule or liquid syrup. Those skilled in the art recognize pharmaceutical binding agents and carriers which protect the agent from degradation in the digestive system and facilitate uptake. Similarly, coatings for the tablet or capsule may be used to ease ingestion thereby encouraging patient compliance. If delivered in liquid suspension, additives may be included which keep the agent suspended, such as sorbitol syrup and the emulsifying agent lecithin, among others, lipophilic additives may be included, such as oily esters, or preservatives may be used to increase shelf life of the agent. Patient compliance may be further enhanced by the addition of flavors, coloring agents or sweeteners. In a related embodiment the agent may be provided in lyophilized form for reconstitution by the patient or his or her caregiver.

The agents described herein may also be delivered via buccal absorption in lozenge form. Similarly, compounds may be included in the formulation which facilitate transepithelial uptake of the agent. These include, among others, bile salts and detergents.

In every case, therapeutic agents destined for administration outside of a clinical setting may be packaged in any suitable way that assures patient compliance with regard to dose and frequency of administration

d) Clinical Trials

Another aspect of current invention involves monitoring the effect of an agent on a treated subject in a clinical trial. In such a trial, the copy number of a foreign polynucleotide, its affinity to cellular transcription factors, the expression or bioactivity of a disrupted gene or polypeptide, or expression or bioactivity of a gene or polypeptide in a disrupted or disruptive pathway, may be used as an indicator of the agent effect on a disease state.

For example, to study the effect of a test agent in a clinical trial, blood may be collected from a subject before, and at different times following treatment with such an agent. The copy number of a foreign polynucleotide may be assayed in monocytes as described above, or the levels of expression of a disrupted gene, such as tissue factor, may be assayed by, for instance, Northern blot analysis, or RT-PCR, as described in this application, or by measuring the concentration of the protein by one of the methods described above. In this way, the copy number, or expression profile of a gene of interest or its mRNA, may serve a surrogate or direct biomarker of treatment efficacy. Accordingly, the response may be determined prior to, and at various times following agent administration. The effects of any therapeutic agent of this invention may be similarly studied if, prior to the study, a suitable surrogate or direct biomarker of efficacy, which is readily assayable, was identified.

### IV. EXAMPLES

Many dietary agents have been identified for their antiviral activities. For instance, many phytochemicals, including the flavonoids, terpenoids, organosulfur compounds, limonoids, lignans, sulphides, polyphenolics, coumarins, saponins, chlorophyllins, furyl compounds, alkaloids, polyines, thiophenes, proteins and peptides have been found to have therapeutic applications against different genetically and functionally diverse viruses. The antiviral mechanism of these agents has been explained on basis of their antioxidant activities, scavenging capacities, inhibition of DNA and RNA synthesis, inhibition of the viral entry, or inhibiting the viral reproduction etc. Large number candidate substances and their antiviral functions have been indentified by a combination of in vitro and in vivo sties using different biological assays. However, no claims have been made on the capacity of these substances to decrease microcompetition between viral and cellular genes, specifically, when the virus is latent or persistent in the host. Furthermore, no claims have been made on the capacity of these substances to decrease the latent viral load, and as a result, decrease the risk developing a disease, or decrease the severity of a current disease, which is developing or was developed during the latent phase. The following section will show examples of dietary agents, including Glycyrrhizic acid/Glycyrrhizin, Quercetin, Epigallocatechin gallate, Cinnamon, Selenium, and Artemisin/Artesunate, with such effects. In these examples treatment with these compounds resulted in a decrease in the concentration of a latent foreign polynucleotide in vitro and in vivo.

### A. Glycyrrhizic Acid (GA)

Licorice, which has been used for thousand of years as a flavoring agent, is derived from the root of Glycyrrhiza glabra. The licorice root contains glycyrrhizic acid (GA), also called glycyrrhizin or glycyrrhizinic acid.

1. KSHV, PEL Cells, Induced Apoptosis

The following in vitro experiments show that GA treatment induced apoptosis of primary effusion lymphoma (PEL) cells that harbor a latent Kaposi sarcoma-associated herpesvirus (KSHV)¹⁴. The apoptosis decreases the number of cells harboring latent foreign polynucleotides in an infected host, decreases the latent viral load in the host, attenuates the microcompetition with cellular genes, decreases the risk of developing clinical symptoms associated with microcompetition-related diseases, and decreases the severity of already existing clinical symptoms of such diseases.

The experiments used twelve different human cell types. Five cell types, BC-1, BC-2, BC-3, BCBL-1, and BCP-1 are B-cells derived from different body cavity-based lymphomas. BC-3, BCBL1, and BCP-1 cells harbor a latent infection with KSHV but not. BC-1 and BC-2 cells harbor both KSHV- and EBV. The examples also used primary human keratinocytes from Clonetics at the second passage. CB33 cells lymphoblastoid cells infected with EBV. Ramos cells (ATCC) are Burkitt lymphoma cells that are negative for KSHV and EBV. SLK are a KS-derived cell line negative for KSHV. KS2616 are cells prepared from a KS lesion of a HIV-negative patient. The KS2616 cells are positive for KSHV.

KSHV latent genes determine the virus persistence. Therefore, Northern blot analysis was used to measure the effect of GA treatment on the expression of three KSHV latent genes in four different KSHV-positive B cells. The there genes were the KSHV latency-associated nuclear antigen 1 (LANA-1, ORF73), the KSHV cyclin protein (v-cyclin, ORF72), and the viral FLICE-inhibitory protein (v-FLIP, K13). All KSHV-infected cells, including PEL cells, express LANA-1. This protein enables the KSHV genome to be present as an episome in latently infected cells. LANA-1 binds to p53 and inhibits the p53-induced apoptosis. LANA-1 also binds the retinoblastoma tumor-suppressor protein (Rb), which possibly inhibits the Rb-induced cell cycle arrest. V-cyclin binds to and activates the cyclin-dependent kinase 6 (cdk6), which leads to phosphorylation and inactivation of p53 and Rb.

The cells were treated with two active and nontoxic GA concentrations (3 and 4 mM). The expressions of the viral gene in treated cells were than compared to those in untreated cells. The example used BC-3 and BCBL-1 cells, which are infected with KSHV, and BC-1 and BC-2 cells, which are co-positive with KSHV and EBV.

On the basis of the growth curves, the BCBL-1 cells were treated with GA for 2 days, the BC-3 cells for 3 days, and BC-2 and BC-1 cells for 6 days. Different times were needed since the cells grow in different rates. The filters were hybridized with a probe specific for LANA-1. This probe detects the LT1 transcripts only. The results showed a dose-dependent decrease of the LT1 transcripts in n all GA-treated cells. Two additional probes that are specific for v-FLIP and v-cyclin were then hybridized. These probes detect the LT2 transcripts. The results showed a decrease in LT1 with an increase in LT2 transcripts. As control the transcript level of β-actin in the GA treated cells were assayed. The results showed similar levels of β-actin in these cells.

The decrease in LANA-1 transcripts in the GA-treated cells might suggest a change in the activity of the LT1/LT2 promoter in these cells. To determine the effect of GA treatment on the LT1/LT2 promoter, BJAB cells were transiently transfected with a reporter gene that expresses luciferase under the control of the LT1/LT2 promoter. The results showed similar level of luciferase expression in untreated and GA-treated cells. These results suggest that GA treatment does not affect the LT1/LT2 promoter activity.

Expression patterns of LANA-1, v-cyclin, and v-FLIP proteins. A Western blot and FACS analysis were used to measure the expression of LANA-1, v-cyclin, and v-FLIP proteins in of BCBL-1, BC-3, and BC-1 cells treated with GA for 2, 3, and 6 days, respectively. Untreated cells positive for KSHV show expression of LANA-1. The GA treatment of the KSHV positive cells decreased LANA-1 expression. The effect was reversible.

FACS analyses of 4 KSHV-positive B cells untreated and following treatment with GA were then used. All KSHV-positive B cells constitutively express v-cyclin. However, following treatment with GA, 35-50% of the cells over-expressed v-cyclin.

The analysis also showed similar expression of v-FLIP in GA treated and untreated cells. It has been showed that the v-FLIP protein blocks Fas-mediated apoptosis in cells latently infected with KSHV. However, in the current experiments GA treatment did not affect apoptosis, which indicates that v-FLIP although expressed, is not interfering with apoptosis.

Previous studies showed that over-expression of v-cyclin promotes apoptosis in cells with elevated levels of cdk6, and that cellular Bcl-2 or v-FLIP does not inhibit this apoptosis. Therefore, the concentration of cdk6 in the BC-1, BC-3, and BCBL-1 cells was assayed. The experiment used normal human lymphocytes, liver cells, 293 human epithelial kidney cells, and BJAB cells as controls. A Western blot analysis revealed that untreated BC-1 and BCBL-1 express high concentrations of cdk6, while untreated BC-3 cells express low concentrations of cdk5. Treatment with GA of these cells increased cdk6 expression 8- to 1-fold, which induced apoptosis in the BC-3 cells. The 293 and BJAB control cells also showed high concentrations of cdk6. However, treatment of the BJAB cells with GA did not result in apoptosis, possibly because these KSHV-negative cells show no expression of v-cyclin. The results suggest that over-expression of the v-cyclin/cdk6 complex might contribute to the apoptosis induced by the GA treatment in the KSHV-positive B cells.

The next step was to examine the biological implications of the modified latent gene expression in the KSHV-positive B cells treated with GA. One of the first intracellular changes during the onset of apoptosis is the disruption of the mitochondrial membrane potential. Proteins that are normally localized in the mitochondrial inter membrane space, such as cytochrome c and AIF, translocate to the nucleus and trigger a cascade of catabolic reactions that result in apoptosis. Following the release from the membrane, cytochrome c with Apaf-1 and procaspase-9 form the “apoptosome.” This complex activates the caspase cascade and apoptosis.

In these experiments, KSHV-positive B cells treated with GA showed the typical disruption of mitochondrial membrane, and many cells with condensed or fragmented chromatin typical of apoptosis (using a TUNEL assay). FACS analysis was used to determine the percentage of TUNEL-positive cells. The analysis showed that the GA treatment induced apoptosis in 80-95% of the KSHV-positive cells. This percentage is higher than the 35-50% of cells over-expressing v-cyclin following GA treatment, indicating that other proteins in addition to v-cyclin induce the observed apoptosis. In comparison to the KSHV-positive cells, uninfected B cells treated with GA showed no disruptions of their mitochondrial membranes or DNA condensation. Treatment with a 12-O-tetradecanoyl-phorbol-13-acetate (TPA), which is know to promote the switch from latent to lytic viral cycle, also did not disrupt the mitochondrial membranes or caused DNA condensation. The lack of the apoptotic effect of the TPA treatment indicates that lytic gene expression was not involvement in the observed apoptosis.

Usually, disruption of the mitochondrial membrane induces caspase-cascade activation and DNA fragmentation. ELISA was used to examine the activation of the caspase cascade in KSHV-positive B cells untreated and following treatment with GA. Them was no activation in any sample, indicating a caspase-independent apoptosis. The experiment then targeted AIF, a mitochondrial oxidoreductase that translocates from the mitochondria to the nucleus under stress condition causing DNA loss and chromatin condensation, typical changes in apoptosis when caspases are inhibited. To identify translocation of AIF, an immunofluorescence analysis was used in KSHV-positive B cells following treatment with GA for 4 days. A cytoplasmic pattern, characteristic of mitochondrial AIF, was evident in the untreated KSHV-positive B cells and in the KSHV-negative cells (BJAB) when untreated and following treatment with. In contrast, the analysis detected a diffuse nuclear staining, indicating translocation to the nucleus, in KSHV-positive B cells following treatment with GA. These observation suggest that GA induced changes in the mitochondrial membrane potential with AIF translocation to the nucleus and DNA fragmentation only in KSHV-positive B cells. To summarize, these observations suggest that the change in the expression of the KSHV latent genes, that is, the decrease in LANA-1 expression and increase in v-cyclin expression, induces the apoptotic effects.

p53 activation and oxidative stress. Several studies showed that over-expression of the v-cyclin protein promotes cell cycle progression and apoptosis. Other studies showed that LANA-1 prevents apoptosis by inactivating p53. A decrease in p53 expression and loss of function characterizes many human malignancies. Following DNA damage, p53 undergoes phosphorylation at Ser15 or Ser20, which induces cell cycle arrest in G₁ and the initiation of DNA repair. If the cell fails to repair the damaged DNA, p53 initiates apoptosis. Therefore, the decrease of LANA-1 expression in KSHV-positive B cells might lead to p53 phosphorylation and apoptosis. To test this idea, the concentration of non-phosphorylated and phosphorylated p53 was assayed in uninfected and KSHV-positive B cells, both untreated and following treatment GA. Following treatment with GA, the experiment detected a high concentration of p53 phosphorylated at Ser15 in KSKV-positive B cells. Then BC-3 cells were transfected with the pLPCX/LANA-1 vector, a mammalian expression vector encoding LANA-1 under the control of the CMV promoter. After 24 hours, the transfected cells were treated with 4 mM GA for 4 days. A Northern blot analysis was used to confirm the presence of the 3.5-kb LANA-1 transcript. A Western blot analysis showed that GA treatment in the presence of high concentrations of LANA-1 resulted in a very low concentration of phosphorylated form of p53 and a high concentration of the non-phosphorylated form of p53. These results confirm that LANA-1 was responsible for the decrease in p53 phosphorylation. These results also support the conclusion that down regulation of LANA-1 by GA was responsible for the increase in p53 phosphorylation.

p53-induces apoptosis is associated with the formation of ROS, including H₂O₂, O₂, and OH. An increase in H₂O₂ increases the concentration of the H₂O₂ scavenger catalase. Catalase activity was assayed in KSHV-positive B cells in untreated cells and following treatment with GA. Following treatment with GA, catalase activity increased 4-fold (BC-3), 2-fold (BCBL-1), and 3-fold (BC-1) in KSHV-positive cells relative to the untreated KSHV-positive cells and relative to the KSHV-negative cells (BJAB) either untreated or following treatment with GA.

FACS was used to determine the distribution of cell cycle in KSHV-positive and uninfected B cells both untreated and following treatment with GA. After 6 days, the treatment with GA caused 99% of KSHV-positive B cells to be blocked in G₁. In contrast, only 25-50% of untreated KSHV-positive cells and uniifected cells were blocked in G₁ when untreated on following treatment with GA. These results indicate that a decrease in LANA-1 expression restores p53 function and induces cell cycle arrest of the latent KSHV-infected cells.

Summary: These results show that GA, while not being toxic at the tested levels, specifically induces apoptosis in latent KSHV-infected cells.

Note that although Curreli, et al. (2005, ibid) showed that GA induces apoptosis in cells carrying a latent infection with KSHV, they did not mention any possible influence of the apoptosis on disease, on microcompetition with foreign DNA, or on the risk of developing a microcompetition-related disease, and the severity of such disease. Specifically, they did not argue against the current misconception that latent infection does not constitute a pathogenic threat (see an expression of such misconception in Babcock 1999¹⁵).

2. EBV, EA Gene, Raji Cells, Inhibition of Persistence Replication

The following in vitro experiment shows what GA treatment inhibits Epstein-Barr virus early antigen (EBV-EA) activation in latently infected cells¹⁶. Since EBV-EA activation is necessary for persistent replication during the latent phase (Prang 1997¹⁷), a decrease in EBV-EA transcripts decreases the latent viral load in the infected host, attenuates the microcompetition with cellular genes, decreases the risk of developing clinical symptoms associated with microcompetition-related diseases, and decreases the severity of already existing clinical symptoms of such diseases.

The example used the Raji cells, a Burkitt-lymphoma-derived cell line that harbors 50 to 60 latent, predominantly extrachromosomal, Epstein-Barr virus genomes (Adams 1987¹⁸). The cells were superinfected with P2HR1 (LS) virus, which results in reactivation of the latent EBV virus and replication in the superinfected cells. Following the superinfection, approximately 95% of the superinfected cells became positive for the EBV early antigen (EA). In the presence of GA, a dose-dependent inhibition of the expression of the EBV-EA was observed. The example also observed a dose-dependent inhibition of viral genome copy number determined by real-time quantitative PCR. The GA concentration required for inhibiting the EBV genome copy number and antigen expression by 50% (EC50) was approximately 5 μM.

### B. Quercetin

Quercetin is a flavonoid and, or more specifically, a flavonol. It is the aglycone form of a number of other flavonoid glycosides, such as rutin and quercitrin, found in citrus fruit, buckwheat and onions. Quercetin forms the glycosides quercitrin and rutin together with rhamnose and rutinose, respectively. Quercetin is classified as IARC group 3 (no evidence of carcinogenicity in humans).

1. EBV, EA Gene, Raji Cells, Inhibition of Persistence Replication

The following in vitro experiment shows that quercetin treatment inhibits EBV-EA activation in latently infected cells¹⁹. Since EBV-EA activation is necessary for persistent replication during the latent phase (Prang 1997, ibid), a decrease in EBV-EA transcripts decreases the latent viral load in the infected host, attenuates the microcompetition with cellular genes, decreases the risk of developing clinical symptoms associated with microcompetition-related diseases, and decreases the severity of already existing clinical symptoms of such diseases.

The experiment used Burkin-lymphoma-derived Raji cells, which harbor 50 to 60 latent, predominantly extrachromosomal Epstein-Barr virus genomes (Adams 1987, ibid). The experiment exposed the Raji cells to EBV-EA positive serum isolated from a patient with nasopharyngeal carcinoma. The serum activated the EBV-EA. Treatment with quercetin derivatives inhibited the EBV-EA activation in the Raji cells without showing cytotoxicity. Quercetin pentaallyl ether (QPA) showed the most significant inhibitory effect on EBV-EA activation (100% inhibition at 1000 mol ratio/TPA and more than 80% inhibition at 500 mol ratio/TPA) and high viability (more than 70% viability at 1000 mol ratio/TPA).

2. HBV, cccDNA, Hep G2.2.15 Cells, Inhibition of Persistence Replication

The following in vitro experiment shows that quercetin treatment decreases the concentration of the Hepatitis B e antigen (HBeAg) in cells latently infected with the Hepatitis B virus (HBV)²⁰. Since a decrease in HBeAg concentration is associated with a decrease in the concentration of the covalently closed circular DNA (cccDNA) form, which is responsible for viral persistence during latency, a decrease in HBeAg indicates a decrease in the latent viral load in the infected host, attenuation of the microcompetition with cellular genes, a decrease in the risk of developing clinical symptoms associated with microcompetition-related diseases, and a decrease in the severity of already existing clinical symptoms of such diseases.

The experiment used the human hepatoma Hep G2.2.15 cell culture system as in vitro model to evaluate the anti-HBV effects of hyperoside, a quercetin derivative (quercetin-3-O-β-D-galactoside). The HBV-producing 2.2.15 cells were obtained from the Institute of Medicinal Biotechnology, Chinese Academy of Medical Sciences (Beijing, China). These cultures were derived from HepG2 cells that were transfected with a plasmid vector containing G418-resistance sequences and 2 head-to-tail dimmers of the HBV genome. The cells were found to produce elevated levels of HBeAg, which is expressed in IBV infected cells during the latent phase (Favre 2003²¹).

The experiment incubated the 2.2.15 cells for 24 hours and then treated them with different concentrations of hyperoside (0.05, 0.025, 0.0125, 0.00625, and 0.003125 g/L) in serum-free medium. The results showed that hyperoside decreased the concentration of HBeAg in the cells. The median effective concentration (IC50) of hyperoside on day 4 was about 0.012 g/L, and on day 8 about 0.009 g/L.

HBV cccDNA is responsible for viral persistence during the natural course of chronic MBV infection and serves as the template for the production of HBV pregenomic RNA (pgRNA), the primary step in HBV replication. A study (Laras 2006²²) used sensitive and specific quantitative real-time polymerase chain reaction (PCR) assays to measure the intrahepatic concentration, pgRNA production, and replicative activity of cccDNA in liver biopsy samples from 34 non-treated patients with chronic hepatitis B (CHB): 12 HBeAg(+) and 22 HBeAg(−). The results showed that in HBeAg(+) patients, the median values of cccDNA and pgRNA levels were 10-fold and 200-fold higher than in HBeAg(−), respectively. These results indicate that a decrease in HBeΛg concentration is associated with a decrease in the concentration of cccDNA, and therefore, a decrease in the HBV DNA copy number during latency. Based on these results, we can conclude that hyperoside treatment decreases the copy number of latent HBV in infected cells.

### C. Epigallocatechin Gallate (EGCG)

Epigallocatechin gallate (EGCG), also known as Epigallocatechin 3-gallate, is a type of catechin and is the most abundant catechin in green tea. It is the ester of epigallocatechol and gallic acid.

1. EBV, Rta Gene, P3HR1 cells, Inhibition of Persistence Replication

The following in vitro experiment shows that EGCG treatment inhibits the activation of the EBV immediate-early protein Rta in latently infected cells²³. Since Rta is essential to for reactivation from latency and maintenance of the latent pool (Pavlova 2003²⁴), a decrease in Rta expression decreases the viral genome copy number in the latently infected cells, decreases the latent viral load in the infected host, attenuates the microcompetition with cellular genes, decreases the risk of developing clinical symptoms associated with microcompetition-related diseases, and decreases the severity of already existing clinical symptoms of such diseases.

P3HR1 is a Burkitt's lymphoma line cell line that carries a latent infection with EBV. A flow cytometry analysis that used immunostaining of Rta with primary antibody and subsequent detection of the primary antibody with fluorescein isothiocyanate (FITC) or rhodamine-conjugated secondary antibody revealed that a low percentage of untreated P3HR1 cells express the Rta protein. The analysis also revealed that following treatment with TSA, a treatment known to activate the EBV lytic cycle, the population of P3HR1 cells expressing Rta increased to 23.4%, and that treatment with 70 mM EGCG decreased the percentage of cells expressing Rta to 9.8%. Treatment with 100 mM EGCG further decreased the percentage of P3HR1 cells expressing Rta to 0.5%. To summarize: EGCG treatment significantly reduced the expression of EBV immediate-early protein Rta, which, in turn, decreases the EBV latent copy number in infected cells.

2. HBV, eccDNA, HepG2-N10 Cells, Inhibition of Persistent Replication

The following in vitro experiment shows that EGCG treatment decreases the copy number of the nuclear covalent closed circular DNA (cccDNA) form, which is characteristic of latent HBV²⁵. In HBV-positive cells, the viral DNA is transported into the nucleus where it transforms into the cccDNA form. Since the cccDNA form is essential for HBV maintenance during latency, a decrease in cccDNA copy number decreases the viral genome copy number in the latently infected cells, decreases the latent viral load in the infected host, attenuates the microcompetition with cellular genes, decreases the risk of developing clinical symptoms associated with microcompetition-related diseases, and decreases the severity of already existing clinical symptoms of such diseases.

The experiment used the human hepatoblastoma cell line HepG2-N10, which was generated by transfecting HepG2 cells with a transfer plasmid which contains a 1.3 unit length of genotype A HBV genome (subtype adw2). Cells were treated with fresh medium containing various concentrations of EGCG. Treatment with a concentration of 22.9 u/ml EGCG reduced the concentration of HBV cccDNA by 60%.

### D. Cinnamaldehyde or Cinnamic Acid

Cinnamic aldehyde or cinnamaldehyde (more precisely trans-cinnamaldehyde) is the chemical compound that gives cinnamon its flavor and odor. Cinnamaldehyde occurs naturally in the bark of cinnamon tees and other species of the genus Cinnamomum like camphor and cassia. These trees are the natural source of cinnamon, and the essential oil of cinnamon bark is about 90% cinnamaldehyde. Most cinnamaldehyde is excreted in urine as cinnamic acid, an oxidized form of cinnamaldehyde.

1. EBV, EA Gene, Raji Cells, Inhibition of Persistent Replication

The following in vitro experiment shows that treatment with cinnamaldehyde or cinnamic acid inhibits EBV-EA activation in latently infected cell 26. Since EBV-EA activation is necessary for persistent replication during the latent phase (Prang 1997, ibid), a decrease in EBV-EA transcripts decreases the latent viral load in the infected host, attenuates the microcompetition with cellular genes, decreases the risk of developing clinical symptoms associated with microcompetition-related diseases, and decreases the severity of already existing clinical symptoms of such diseases.

The experiment used the Raji cells, a Burkitt-lymphoma-derived cell line that harbors 50 to 60 latent, predominantly extrachromosomal, Epstein-Barr virus genomes (Adams 1987, ibid). The cell were treated with 2-O-tetradecanoylphorbol-13-acetate (TPA), known to promote activation of EBV-EA. The cells were treated with cinnamaldehyde or cinnamic acid and an indirect immunofluorescence technique was used to stain the EBV-EA expressing cells. The inhibition activity of the test compound was estimated by the percentage of positive cells compared to controls. The results showed that cinnamaldehyde and cinnamic acid inhibited EBV-EA activation in a dose dependent manner. The IC₅₀ of cinnamaldehyde or cinnamic acid was 158 and 40, respectively. IC₅₀ represents the mol ratio to TPA that inhibits 50% of positive controls (100%) activated with 32 pmol TPA.

### E. Selenium (Se)

Selenium is a chemical element with the atomic number 34, represented by the chemical symbol Se, and an atomic mass of 78.96. Selenium is a semi metal that rarely occurs in its elemental state in nature. It is toxic in large amounts, but trace amounts of it are necessary for normal cellular function in most, if not all, animals, forming the active center of the enzymes glutathione peroxidase and thioredoxin reductase and three known deiodinase enzymes.

1. EBV, EA Gene, Raji Cells, Inhibition of Persistent Replication

The following in vitro experiment shows that treatment with Se inhibits EBV-EA activation in latently infected cells²⁷. Since EBV-EA activation is necessary for persistent replication during the latent phase (Prang 1997, ibid), a decrease in EBV-EA transcripts decreases the latent viral load in the infected host, attenuates the microcompetition with cellular genes, decreases the risk of developing clinical symptoms associated with microcompetition-related diseases, and decreases the severity of already existing clinical symptoms of such diseases.

The experiment used the Raji cells, a Burkitt-lymphoma-derived cell line that harbors 50 to 60 latent, predominantly extrachromosomal, Epstein-Barr virus genomes (Adams 1987, ibid). The experiment stimulated the Raji cells with butyrate and croton oil. The stimulated cells were incubated with Se-rich rice extract. The experiment than used the indirect immunological flurescence method to count the EBV-EA positive expression rate and the inhibition rate. The results showed that Se-rich rice extract significantly inhibited the EBV-EA expression in Raji cells. At extract concentrations of 0.016, 0.078, and 0.388 μg/ml, the inhibition rate of EA expression was 2.85%, 12.88%, and 20.75%, respectively.

### F. Artemisinin/Artesunate

The compound (a sesquiterpene lactone) is isolated from the plant Artemisia annua. Artesunate is a derivative of artemisinin. Artemisia has been used by Chinese herbalists for more than a thousand years in the treatment of malaria.

1. CMV, Decreased of Latent Viral Load, Transplant Patient

The following in vivo study shows that treatment with artesunate decreases the DNA viral load of CMV in a patient when initiated 120 days post infection²⁸. Since it takes the CMV about 4 weeks (120 days) to establish latency in the human body (Schroeder 2004²⁹), we can conclude that the patient in this study was already at the latent phase by the time the treatment with artesunate was initiated, and that the treatment decreased the latent viral load of CMV in his body. In general, this study shows that treatment with artesunate decreases the latent viral load of foreign DNA in an infected host and therefore, attenuates the microcompetition with cellular genes, decreases the risk of developing clinical symptoms associated with microcompetition-related diseases, and decreases the severity of already existing clinical symptoms of such diseases.

The patient in this study was a 12-year-old boy with X-linked adrenoleukodystrophy who received haploidentical T cell-depleted hematopoietic stem cells from his father. Starring from day 15 after transplantation, CMV virernia was noted. After 120 days of conventional treatments, the viral DNA load increased to 1.15*10̂6 copies/mL. At this point, oral treatment with artesunate (100 mg/day) was initiated. Results showed a favorable response with rapid reduction in viral load and improved hematopoiesis within 10 days. By day 7, the viral DNA load showed a 1.7-2.1 log decrease. The extent of the response was similar to the response the study observed during the initial ganciclovir treatment administered to the patient (1-log reduction by day 7), and to the response to ganciclovir and foscarnet treatments reported in Emery 1999³⁰. Furthermore, the CMV load kinetics during artesunate treatment showed a short viral decay (T ½ 0.9-1.9 days), similar to the kinetics of ganciclovir therapy also reported in Emery 1999 (ibid). In addition, no adverse effects were observed during the first 30 days of artesunate treatment, and no increase in viremia for 76 days after completion of therapy.

### V. PREFERRED EMBODIMENTS

The examples showed that GA, quercetin, EGCG, cinnamaldehyde or cinnamic acid, selenium, and artesunate, decrease the latent viral load in an infected host. This decrease will be apparent in any method capable of measuring this viral load, directly or indirectly, including, but not limited to real time PCR, and serology. Moreover, this decrease indicates that the dietary supplements can attenuate microcompetition between foreign DNA and cellular genes, and decrease the risk of developing clinical symptoms, or the severity of already existing clinical symptoms associated with such microcompetition. Therefore, a preferred embodiment of the current invention is an effective dose of one of these dietary supplements, or other dietary supplements that can serve a source for these dietary supplement, such as licorice, which can serve as a source of GA, green tea, which can serve as a source of EGCG, cinnamon, which can serve as a source of cinnamaldehyde or cinnamic acid, etc. In addition, a preferred embodiment of the current invention is any combination of these dietary supplements, for example, a capsule that include licorice, quercetin, green tea, cinnamon, and selenium.

While the above describes what are presently believed to be the preferred embodiments of the invention, those skilled in the art will realize that changes and modifications may be made thereto without departing from the spirit of the invention. It is intended to claim all such changes and modifications that fall within the true scope of the invention.

### REFERENCES

1 H Polansky. Microcompetition with Foreign DNA and the Origin of Chronic Disease, CBCD Publishing, 543 p, 2003.
2 H Polansky. Assays for drug discovery based on microcompetition with a foreign polynucleotide. U.S. Pat. No. 7,381,526, Jun. 3, 2008.
³ H Polarsky. Dietary supplements for decreasing the latent foreign DNA load. Patent application Ser. No. 12/335,576.
⁴Besson C, Amiel C, Le-Pendeven C, Brice P, Fermé C, Carde P, Hermine O, Raphael M, Abel L, Nicolas J C. Positive correlation between Epstein-Bar virus viral load and anti-viral capsid immunoglobulin G titers determined for Hodgkin's lymphoma patients and their relatives. J Clin Microbiol. January 2006;44(1):47-50.
⁵ Stowe R P, Kozlova E V, Yetman D L, Walling D M, Goodwin J S, Glaser R. Chronic herpesvirus reactivation occurs in aging. Exp Gerontol. June 2007;42(6):563-70. Epub Jan. 30, 2007.
⁶ Burbelo P D, Meoli E, Leahy H P, Graham J, Yao K, Oh U, Janik J E, Mahieux R, Kashanchi F, Iadarola M J, Jacobson S. Anti-HTLV antibody profiling reveals an antibody signature for HTLV-I-associated myelopathy/tropical spastic paraparesis (HAM/TSP). Retrovirology. Oct. 20, 2008;5:96.
7 Gonelli A, Boccia S, Boni M, Pozzoli A, Rizzo C, Querzoli P, Cassai E, Di Luca D. Human herpesvirus 7 is latent in gastric mucosa. J Med Virol. April 2001;63(4):277-83.
8 Smith R L, Morroni J, Wilcox C L. Lack of effect of treatment with penciclovir or acyclovir on the establishment of latent HSV-I in primary sensory neurons in culture. Antiviral Res. October 2001;52(1):19-24.
9 Young L S, Dawson C W, Eliopoulos A G. The expression and function of Epstein-Barr virus encoded latent genes. Mol Pathol. October 2000;53(5):238-47.

10 Virgin H W. Immune regulation of viral infection and vice versa. Immunol Res. 2005;32(1-3):293-315. Review.
¹¹ Gangappa S, Kapadia S B, Speck S H, Virgin H W 4th. Antibody to a lytic cycle viral protein decreases gammaherpesvirus latency in B-cell-deficient mice. J Virol. November 2002;76(22):11460-8.
¹²Hoshino Y, Qin J, Follmann D, Cohen J I, Straus S E. The number of herpes simplex virus-infected neurons and the number of viral genome copies per neuron correlate with the latent viral load in ganglia Virology. Mar. 1, 2008;372(1):56-63. Epub Nov. 28, 2007.
¹³ Kim I J, Flañ E, Woodland D L, Blackman M A. Antibody-mediated control of persistent gamma-herpesvirus infection. J Immunol. Apr. 15, 2002;168(8):3958-64.
14 Curreli F, Alvin E. Friedman-Kien A E, Flore O. Glycyrrhizic acid alters Kaposi sarcoma-associated herpesvirus latency, triggering p53-mediated apoptosis in transformed B lymphocytes. J Clin Invest. Mar. 1, 2005; 115(3):642-652.
¹⁵ Babcock G J, Decker L L, Freeman R B, Thorley-Lawson D A. Epstein-barr virus-infected resting memory B cells, not proliferating lymphoblasts, accumulate in the peripheral blood of immunosuppressed patients. J Exp Med. Aug. 16, 1999;190(4):567-76.
16 Lin J C, Cherng J M, Hung M S, Baltina L A, Baltina L, Kondratenko R. Inhibitory effects of some derivatives of glycyrrhizic acid against Epstein-Barr virus infection: structure-activity relationships. Antiviral Res. July 2008;79(1):6-11. Epub Mar. 31, 2008.
17 Prang N S, Hornef M W, Jager M, Wagner H J, Wolf H, Schwarzmann F M. Lytic replication of Epstein-Barr virus in the peripheral blood: analysis of viral gene expression in B lymphocytes during infectious mononucleosis and in the normal carrier state. Blood. Mar. 1, 1997;89(5):1665-77.
18 Adams A. Replication of latent Epstein-Barr virus genomes in Raji cells. J Virol. May 1987; 61(5):1743-1746.
19 Iwase Y, Takemura Y, Ju-ichi M, Mu a T, Ichiisbi F, Ito C, Furukawa H, Yano M, Tokuda H, Nishino H. Inhibitory effect of flavonoid derivatives on Epstein-Barr virus activation and two-stage carcinogenesis of skin tumors. Cancer Lett. Nov. 28, 2001;173(2):105-9.
20 Wu L L, Yang X B, Huang Z M, Liu H Z, Wu G X. In vivo and in vitro antiviral activity of hyperoside extracted from Abelmoschus manihot (L) medik. Acta Pharmacol Sin. March 2007;28(3):404-9.
21 Favre D, Petit M A, Trépo C. Latent hepatitis B virus (HBV) infection and HBV DNA integration is associated with further transformation of hepatoma cells in vitro. ALTEX. 2003;20(3):131-42.
22 Laras A, Koskinas J, Dimou E, Kostamena A, Hadziyannis S J. Intrahepatic levels and replicative activity of covalently closed circular hepatitis B virus DNA in chronically infected patients. Hepatology. September 2004;44(3):694-702.
23 Chang L K, Wei T T, Chiu Y F, Tung C P, Chuang J Y, Hung S K, Li C, Liu S T. Inhibition of Epstein-Barr virus lytic cycle by (−)-epigallocatechin gallate. Biochem Biophys Res Commun. Feb. 21, 2003;301(4):1062-8.
24 Pavlova I V, Virgin H W 4th, Speck S H. Disruption of gammaherpesvirus 68 gene 50 demonstrates that Rta is essential for virus replication. J Virol. May 2003;77(10):5731-9
25 Xu J, Wang J, Deng F, Hu Z, Wang H. Green tea extract and its major component epigallocatechin gallate inhibits hepatitis B virus in vitro. Antiviral Res. June 2008;78(3):242-9. Epub Feb. 8, 2008.
26 Motohashi N, Yamagami C, Tokuda H, Okuda Y, Ichiishi E, Mukainaka T, Nishino H, Saito Y. Structure-activity relationship in potentially anti-tumor promoting benzalacetone derivatives, as assayed by the epstein-barr virus early antigen activation. Mutat Res. Jan. 24, 2000;464(2):247-54.
27 Jian S W, Mei C E, Liang Y N, Li D, Chen Q L, Luo H L, Li Y Q, Cai T Y. [Influence of selenium-rich rice on transformation of umbilical blood B lymphocytes by Epstein-Barr virus and Epstein-Barr virus early antigen expression]. Ai Zheng. January 2003;22(1):26-9. [Article in Chinese]
28 Shapira M Y, Resnick I B, Chou S, Neumann A U, Lurain N S, Stamminger T, Caplan O, Saleh N, Efferth T, Marschall M, Wolf D G. Artesunate as a potent antiviral agent in a patient with late drug-resistant cytomegalovirus infection after hematopoietic stem cell transplantation. Clin Infect Dis. May 1, 2008;46(9):1455-7.
29 Schroeder R, Michelon T, Fagundes I, Bortolotto A, Lammerhirt E, Oliveira J, Santos A, Bittar A, Keitel E, Garcia V, Neumann J, Saitovitch D. Cytomegalovirus disease latent and active infection rates during the first trimester after kidney transplantation. Transplant Proc. May 2004;36(4):896-8.
30 Emery V C, Cope A V, Bowen E F, Gor D, Griffiths P D. The dynamics of human cytomegalovirus replication in vivo. J Exp Med 1999; 190:17782.

What is claimed is:

1. A method for treating an animal or human subject, the method comprising the steps of.
a. Selecting an agent, wherein said agent is a dietary supplement;
b. Administering said agent to said subject to decrease the concentration of an antibody elicited by a foreign polynucleotide latent in said subject, or an antibody elicited by a polypeptide expressed by said polynucleotide, or the polynucleotide load in the subject.

2. The method in claim 1, wherein said latent foreign polynucleotide is a latent or persistent virus.

3. The method in claim 2, wherein said virus is selected from the group consisting of Epstein Barr Virus (E,BV), Cytomegalovirus (CMV), Herpes Simplex Virus 1 (HSV-1), Herpes Simplex Virus 2 (HSV-2), Human Papillomavirus (HPV), Adenovirus, Kaposi's Sarcoma-Associated Herpesvirus (Huma Herpesvirus 8), Hepatitis B, Torquetenovirus (ITV), JC Virus (JCV), and BK Virus (BKV).

4. The method in claim 1, wherein said subject is suffering from a chronic disease, or is at risk of developing a chronic disease.

5. The method in claim 4, wherein said chronic disease is selected from the group consisting or atherosclerosis, cancer, obesity, ostcoarthritis, type II diabetes, type I diabetes, multiple sclerosis, asthma, lupus, thyroiditis, inflammatory bowel disease, rheumatoid arthritis, psoriasis, atopic dermatitis, graft versus host disease, and other autoimmune diseases.

6. The method in claim 1, wherein said agent is selected from the group consisting of licorice, glycyrrhizic acid, quercetin, green tea, epigallocatechin gailate, cinnamon, cinnamaldehyde, cinnamic acid, selenium, artemisinin, and artesunate, and any combination thereof.

7. The method in claim 1, wherein said agent is consists of the combination of licorice, quercetin, green tea, cinnamon, and selenium.
